(19) United States
(12) Patent Application Publication
(10) Pub. No.: US 2021/0379049 A1
(43) Pub. Date: Dec. 9, 2021

(54) PPAR-gamma AGONIST FOR TREATMENT OF BLOOD CANCERS

(71) Applicant: Coherus Biosciences, Inc, Redwood, CA (US)

(72) Inventor: Christos Mantzoros, Watertown, MA (US)

(21) Appl. No.: 16/326,018

(22) PCT Filed: Aug. 18, 2017
(86) PCT No.: PCT/US2017/047578

§ 371 Date: Feb. 15, 2019

Related U.S. Application Data
(60) Provisional application No. 62/376,749, filed on Aug. 18, 2016.

(51) Int. Cl. A61K 31/47; A61P 35/02

(52) U.S. Cl. CPC A61K 31/47; A61P 35/02

(57) ABSTRACT

Methods of treatment of blood cancers including leukemia and myeloma with the compound of formula (I) known as INT131:

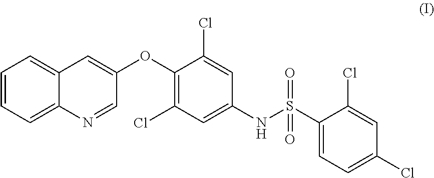 [SMILES: O=S(=O)(Nc1cc(Cl)c(Oc2cnc3ccccc3c2)c(Cl)c1)c1ccc(Cl)cc1Cl]

### FIELD OF THE INVENTION

The present invention relates to methods of treatment of blood cancers including leukemia and myeloma.

### BACKGROUND OF THE INVENTION

Blood cancers result from overproduction of dysfunctional blood cells. There are three main types of blood cancers called leukemia, lymphoma and myeloma. Over 170,000 people in the United States and 700,000 people worldwide are diagnosed with some form of blood cancer each year.

Leukemia is a cancer of the precursor cells to white blood cells (“WBC,” i.e. leukocytes), which most commonly reside in the bone marrow. By nature, leukemia is malignant and results in malformed white blood cells that inhibit production of normal blood cells and can easily spread to various organs causing reduced function and failure. There are several types of leukemia that can be divided by the type of WBC, that is affected and the speed by which the disease progresses.

Leukemia specifically affects either lymphoid or myeloid-type precursor cells and thus are named either lymphoblastic (a.k.a. lymphocytic) or myelogenous leukemia. Leukemia may also progress quickly or slowly and as such are divided into either acute or chronic leukemia. Thus, there are four main types of leukemia known as acute myelogenous leukemia (“AML”), chronic myelogenous leukemia (“CML”), acute lymphoblastic leukemia (“ALL”), and chronic lymphocytic leukemia (“CLL”). Other rarer types of leukemia also exist such as hairy cell leukemia. Further divisions can be made based on the age of the patient such as juvenile and adult leukemia. Worldwide, around 352,000 people are diagnosed with leukemia every year. Around 62,000 of those people live in the United States.

CLL is the most common type of leukemia and is most common in people over 60 years old and is rare in people under 40. CLL is the result of an overabundance of abnormal B lymphocytes. This buildup occurs because the abnormal B lymphocytes live longer than an average B lymphocyte and thus over time there are an obtrusive number of dysfunctional B lymphocytes. These dysfunctional B lymphocytes do not protect against infection and also result in fewer red blood cells leading to anemia, fewer platelets leading to blood clotting problems, and fewer normal WBCs leading to serious infections.

ALL can occur in adults or children. ALL differs from CLL because ALL is the result of an overproduction of abnormal B lymphoblasts rather than lymphocytes. As the name implies, these lymphoblasts are capable of dividing on their own, which results in an exponential increase in the number of abnormal B lymphoblasts. The results of this overabundance of dysfunctional B lymphoblasts are similar to those for CLL, however, because of the rapid growth rate of the disease, an affected individual may only live for a few months if not diagnosed and treated.

Like CLL, CML occurs most often in adults. CML is the results of an overabundance of abnormal myeloid cells including neutrophils, eosinophils, basophils and their precursor cells. CIVIL presents with similar symptoms as other types of leukemia, which include fatigue, weight loss, dizziness, fever, frequent bleeding or bruising, frequent infections, night sweats and loss of appetite.

AML, like CML, is the result of an overabundance of abnormal myeloid cells. Like, ALL, AML progresses rapidly due to the exponential growth of the dysfunctional cells. Current treatment options for AML include watchful waiting, radiation, chemotherapy, antibodies, stem cell transplantation and surgery. These treatment options are also available for other types of leukemia.

Myeloma is a cancer of B plasma cells, a specialized type of B lymphocyte that normally lives liar only a few days and secretes antibodies. Myeloma comes in a few types characterized by their location including, predominantly, multiple myeloma and also localized myeloma, plasmacytoma and extramedullary myeloma. Multiple myeloma is a B plasma cell cancer that occurs simultaneously in several different regions. Plasmacytoma is a B plasma cell cancer that occurs as a tumor in a specific area. Localized myeloma is a B plasma cell cancer that occurs in a specific area but includes some parts of neighboring areas. Extramedullary myeloma is a B plasma cell cancer that occurs in areas other than the bone marrow such as skin, muscles and lungs.

Adiponectin circulates in the blood stream and exhibits beneficial effects including anti-inflammatory, antiproliferative, and proapoptotic properties. Akl H K et al., Role of adiponectin in chronic lymphocytic leukemia, Egyptian J Haematology, 2012, 37(4), 187-192. Serum adiponectin levels are reduced in AML and ALL patients compared to non-cancer patients of similar age, sex and body mass index and are thought to be a possible biomarker of leukemia. Aref S et al., Impact of serum adiponectin and leptin levels in acute leukemia, Hematology, 2013 July 18(4):198-203. Adiponectin has also been found to be an antiangiogenic factor in CLL. Molica S et al., Does adiponectin act as an antiangiogenic factor in B-cell chronic lymphocytic leukemia?, Adv Hematol. 2009, 2009:287974. Further, treatment of CML with interferon was found to suppress inflammatory cytokines and increase adiponectin levels indicating that adiponectin levels may not only be a biomarker for leukemia but may also be a treatment. Ferit A, et al., Plasma Adiponectin Concentrations in Relation to Chronic Lymphocytic Leukemia and Chronic Myeloproliferative Diseases, Blood, 2004, 104(11), 4743.

Decreased serum adiponectin levels are associated with a higher risk of multiple myeloma in overweight and obese people. Hofmann J. N, et al., Low Levels of Circulating Adiponectin Are Associated with Multiple Myeloma Risk in Overweight and Obese Individuals, Cancer Res, 2016 Apr. 1, 76(7), 1935-1941. Further, exogenous adiponectin was found to induce myeloma cell death in the same mouse model. Fowler J A, et al., Host-derived adiponectin is tumor-suppressive and a novel therapeutic target for multiple myeloma and the associated bone disease, Blood, 2011 Nov. 24, 118(22), 5872-5882.

Despite the various treatment options and plethora of drugs that have been approved for use as a chemotherapeutic agent for leukemia and myeloma, the five-year survival rate in the United States is only around 60% for leukemia and around 50% for myeloma. Thus, there is a need in the art for development of further treatments for leukemia.

INT131 (also known as CHS-131) is a novel, first-in-class, selective modulator of peroxisome proliferator-activated receptor gamma (PPARγ). The PPARγ is a transcription factor belonging to the steroid/thyroid/retinoid receptor superfamily. To date, PPARγ agonists have been therapeutic agents for disorders such as obesity, diabetes and dyslipidemia.

INT131 is structurally different from other PPARγ agonists. INT131 lacks the TZD (glitazone) scaffold of rosiglitazone and pioglitazone. Therefore, INT131 binds the AF2 (transcriptional activation function 2) helix without contacting helix 12. As a result, INT131 selectively activates PPARγ functions.

PPARγ protein function regulates target gene transcription in a ligand-dependent, cofactor-dependent manner by differential co-factor/co-repressor recruitment. As a result of these complex combinatorial chemistry mechanisms, and the unique structure of INT131, the effects of selective activation of PPARγ is difficult to predict. For instance, it has been shown that subjects who are administered INT131 lack TZD-induced adverse events. Therefore, transcriptional activation effected by INT131 differs from other PPARγ agonists. As a result, the effect of other PPARγ agonists on patients is not predictive of the utility of INT131.

### SUMMARY OF THE INVENTION

It has now been discovered that the PPARγ agonist INT131 (also known as CHS-131) is effective for treating blood cancers including leukemia and myeloma.

In one aspect, the present invention provides methods of treating leukemia or myeloma and symptoms thereof. The methods typically involve administering to a subject in need thereof a therapeutically effective amount of compound INT131 described in U.S. Pat. No. 7,601,841. INT131 is unique among PPARγ agonists in that it is a selective activator of a highly limited number of PPARγ pathways. Among these INT131-sensitive pathways are metabolic pathways including those pathways regulated by the hormone adiponectin.

As a result of this selective activation, administration of INT131 to patients results in fewer side effects than administration of other PPARγ agonists. For example, INT131 was equally efficacious in reducing HbAlc levels as 45 mg of pioglitazone but subjects taking INT131 experienced less edema, weight gain, and hemodilution than those taking pioglitazone. See, DePaoli, et al. Diabetes Care, 2014 July; 37(7):1918-23. INT131 can administered to treat blood cancers while limiting side effects. Limiting side effects is advantageous as it helps preserve the quality of life for subject taking the medication and results in unproved subject compliance with taking medication.

### BRIEF DESCRIPTION OF THE DRAWINGS

FIG. 1 is a bar graph of levels of adiponectin following administration of INT131

### DETAILED DESCRIPTION OF THE INVENTION

In particular, the compound (I),

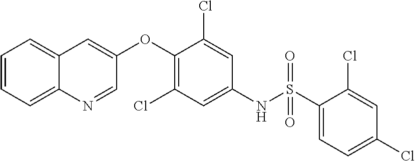 [SMILES: O=S(=O)(Nc1cc(Cl)c(Oc2cnc3ccccc3c2)c(Cl)c1)c1ccc(Cl)cc1Cl]

has been found to be unexpectedly effective for blood cancer including leukemia and myeloma.

This compound is also known as INT131 and CHS-131.

### Definitions

The terms “treat”, “treating” and “treatment” refer to a method of alleviating or abrogating a disease and/or its attendant symptoms.

The term “therapeutically effective amount” refers to that amount of the compound being administered sufficient to prevent development of or alleviate to some extent one or more of the symptoms of the condition or disorder being treated.

The term “subject” is defined herein to include animals such as mammals, including but not limited to, primates (e.g., humans), cows, sheep, goats, horses, dogs, cats, rabbits, rats, mice and the like. In preferred embodiments, the subject is a human.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either net or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either net or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isbutyric, oxalic, maleic, malonic, benzoic, succinic, suberic, fumeric mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present inventions contain both basic and acidic functionalities that allow the compounds to be converted into either base or acid addition salts.

The neutral forms of the compounds may be registered by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In additional to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, be bioavailable by oral administration whereas the parent drug is not. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity. Additional examples include peptidyl derivatives of a compound of the invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

### EMBODIMENTS OF THE INVENTION

A new use of a known compound that modulate PPARγ has now been discovered.

The present invention is further directed to a method of treating a blood cancer selected from leukemia and myeloma or their symptoms in a subject in need thereof comprising administering to the subject a pharmaceutical composition comprising a therapeutically effective amount of INT131 or a pharmaceutically acceptable salt, prodrug, or isomer thereof.

In one embodiment, the leukemia is selected from the group consisting of acute myelogenous leukemia, chronic myelogenous leukemia, acute lymphocytic leukemia, chronic lymphocytic leukemia, hairy cell leukemia, myelodysplastic syndromes and myeloproliferative disorders.

In another embodiment, the myeloma is selected from the group consisting of multiple myeloma (including relapsed or refractory multiple myeloma), localized myeloma, plasmacytoma and extramedullary myeloma.

In another embodiment, INT131 is in the form of a besylate salt.

In another embodiment, the therapeutically effective amount is from about 0.1 to about 10 milligrams, preferably from about 0.5 to about 5 milligrams and more preferably from about 1 to about 3 milligrams. In another embodiment, the therapeutically effective amount is at least about 0.5 milligrams, about 1 milligrams, about 2 milligrams, about 3 milligrams, about 4 milligrams, about 5 milligrams, about 6 milligrams, about 7 milligrams, 8 milligrams, about 9 milligrams or about 10 milligrams.

In another embodiment, a composition comprising a therapeutically effective amount of INT131 is administered to a subject in need thereof at an interval that includes, but is not limited to, twice a day, daily, every other day, three times a week, twice a week, weekly, every other week, twice a month, monthly, and every other month.

In one embodiment, administration of INT131 improves overall survival as compared to placebo or a standard of care for the blood cancer. In another embodiment, administration of INT131 results in overall response rate (ORR) indicating treatment according to the 2008 Modified International Workshop on Chronic Lymphocytic Leukemia (IWCLL) National Cancer Institute-sponsored Working Group (NCI-WG) Guidelines for Tumor Response. In another embodiment, administration if INT131 results in improved progression-free survival (PFS) as compared to placebo or a standard of care for the blood cancer.

In one embodiment, administration of INT131 results in increased adiponectin levels in a subject with a blood cancer. In another embodiment, administration of INT131 to a subject with a blood cancer results in increased adiponectin levels and treatment of the blood cancer. In yet another embodiment, the adiponectin level in a subject with a blood cancer increased by administration of INT131 to the subject. In still another embodiment, a subject with a blood cancer is treated by administration of INT131 to increase adiponectin levels in the subject. In another embodiment, a subject with a blood cancer is treated by increasing adiponectin levels wherein adiponectin levels are increased by administration of INT131.

In another embodiment, a composition comprising a therapeutically effective amount of INT131 is administered to orally to a subject. In yet another embodiment, the composition is substantially the same as those disclosed in US Publication 2013-0243865, the disclosure of which is expressly incorporated herein by reference.

### EXAMPLES

### Example 1

INT131 is a Potent Upregulator of Adiponectin in Patients with Reduced Adiponectin Levels

### Method

A randomized, double-blind, placebo-controlled, 24-week study was conducted in which adiponectin levels were measured. The study had a 2-week lead-in period, a 24-week double-blind treatment period and a 2-week follow up period. 367 subjects with type 2 diabetes (TD2)—a disease in which patient adiponectin levels are reduced-were randomly assigned to receive either 0.5, 1, 2 or 3 milligrams (“mg”) of INT131 besylate, 45 mg of pioglitazone or placebo daily for 24 weeks. To measure adiponectin levels blood was drawn at Weeks 0, 2, 6, 12 and 24.

The results of this study demonstrated that 1, 2, and 3 mg doses of INT131 caused a statistically significant reduction of HbA_{1c} levels as compared to placebo. Further, the study demonstrated that the 2 and 3 mg doses of INT131 reduced HbA_{1c} levels at least as well as 45 mg of pioglitazone, which is an FDA approved treatment for TD2. See, DePaoli, et al. Diabetes Care 2014; 37:1918-1923. Thus, 2 and 3 mg doses of INT131 would be effective in treating TD2.

### Adiponectin Results

At baseline (Week 0) mean adiponectin levels were 1.94 micrograms per milliliter (“μg/mL”). The mean adiponectin levels at baseline and Week 24, and the mean change in adiponectin levels from baseline (Week 0) to Week 24 are disclosed in Table 1, below. The standard deviation for samples tested in each group is listed in (parenthesis). Mean baseline adiponectin values were similar for the treatment groups.

TABLE 1
Changes in Adiponectin Serum Levels

| Mean | | | | | | 45 mg |
|---|---|---|---|---|---|---|
| Adiponectin | | 0.5 mg | 1 mg | 2 mg | 3 mg | Pioglit- |
| (μg/mL) | Placebo | INT131 | INT131 | INT131 | INT131 | azone |
| n | 56 | 56 | 59 | 60 | 60 | 57 |
| Week 0 | 1.85 | 1.73 | 1.87 | 1.87 | 2.00 | 2.32 |
| | (1.153) | (1.190) | (1.217) | (1.098) | (1.215) | (2.185) |
| Week 24 | 1.9 | 2.28 | 3.15 | 5.14 | 5.83 | 5.28 |
| | (1.510) | (1.540) | (2.533) | (3.650) | (4.826) | (3.222) |
| Mean | 0.05 | 0.56 | 1.28 | 3.27 | 3.83 | 2.96 |
| Change | (0.680) | (0.906) | (1.882) | (3.002) | (4.313) | (2.618) |

The treatment comparisons of 1 mg, 2 mg, and 3 mg doses of INT131 with placebo were statistically significant (p≤0.0109). This demonstrates that treatment with INT131 resulted in a statistically significant increase in adiponectin levels in patients suffering from a disease in which adiponectin levels are reduced (e.g. TD2). Thus, INT131 is therapeutically effective in treating patients with diseases (e.g. blood cancers) in which adiponectin levels are reduced.

Additionally, the treatment comparisons of 0.5 mg, 1 mg, and 3 mg doses of INT131 with pioglitazone 45 mg were statistically significant (p≤0.0408). Thus, the dose dependent increase of adiponectin levels by INT131 is independent from the increase resulting from pioglitazone.

### CONCLUSIONS

The effect of treatment on serum adiponectin was assessed, enabling a more direct comparison of the relative potencies of INT131 and pioglitazone 45 mg as selective PPARγ modulators. The mean change in adiponectin from baseline to Week 24 with LOCF (last observation carried forward) was 0.05 sg/mL for the placebo group, 0.56 sg/mL for the INT131 0.5 mg group, 1.28 μg/mL for the INT131 1 mg group, 3.27 μg/mL for the 2 mg group, 3.83 μg/mL for the INT131 3 mg group, and 2.96 μg/mL for the pioglitazone 45 mg group. Therefore, in a manner quantitatively different from the effects on HbA_{1c}, where the INT131 dose roughly equivalent to pioglitazone 45 mg is between 2 mg and 3 mg, a dose of INT131 between 1 mg and 2 mg was equivalent to pioglitazone 45 mg for increasing adiponectin levels.

Surprisingly, administration of INT131 at either 2 or 3 mg resulted in a greater upregulation of serum adiponectin levels than did administration of at least 22 times the amount of pioglitazone. Small amounts of INT131 are at least as efficacious in treating diseases in which adiponectin levels are reduced as are other drugs which also increase adiponectin levels.

Administration of 1, 2, or 3 mg of INT131 treats patients suffering from diseases in which adiponectin levels are reduced.

### Example 2

### INT131 is a Potent Upregulator of Adiponectin in Healthy Subjects

### Method

A study was conducted to determine the effect of INT131 on serum adiponectin levels. Thirty healthy subjects were randomly selected to receive either placebo, 0.1 mg INT131, 1 mg INT131 or 4 mg INT131 daily for 14 days. To measure adiponectin levels blood was drawn at Days 1, 4, 8 and 14.

### Results

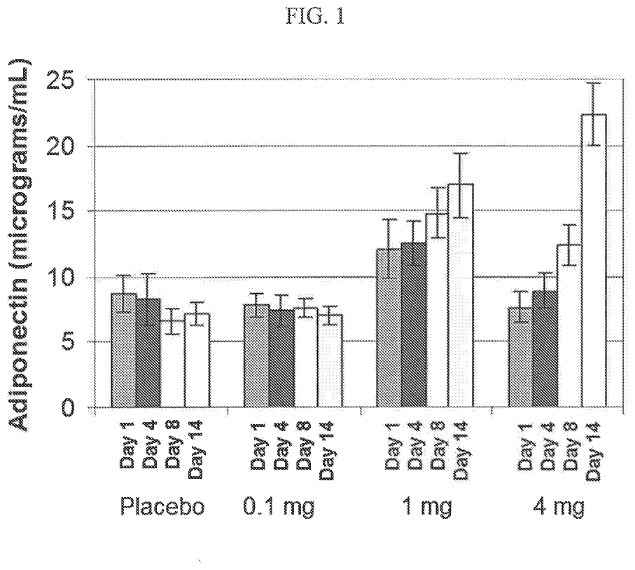

From Day 1 to Day 14 administration of placebo and 0.1 mg INT131 resulted in no significant change in serum adiponectin levels and further administration of 0.1 mg INT131 resulted in no significant change in adiponectin levels over placebo. See FIG. 1. However, administration of 1 mg or 4 mg INT131 resulted in a significant change in serum adiponectin levels over placebo and a significant change from Day 1 to Day 14. Thus, administration of INT131 is capable of upregulating adiponectin in healthy individuals.

INT131 is potent upregulator of adiponectin, whether or not a subject suffers from TD2 or any disease in which adiponectin levels are reduced. As a result, INT131 increases adiponectin levels in all subjects and is especially useful in the treatment of any disease in which adiponectin levels are reduced. Therefore, INT131 is effective in treating blood cancers (e.g. leukemia and myeloma) since these individuals suffering from these diseases have reduced adiponectin levels.

What is claimed is:

1. A method of treating a blood cancer selected from leukemia and myeloma in a subject in need thereof comprising administering to the subject a pharmaceutical composition comprising a therapeutically effective amount of a compound of formula (I), 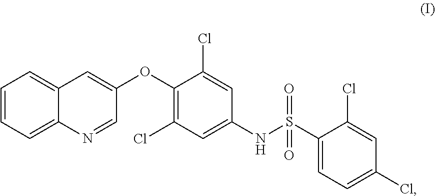 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

2. The method of claim 1, wherein the blood cancer is leukemia.

3. The method of claim 2, wherein the leukemia is selected from the group consisting of acute myelogenous leukemia, chronic myelogenous leukemia, acute lymphocytic leukemia, chronic lymphocytic leukemia, hairy cell leukemia, myelodysplastic syndromes and myeloproliferative disorders.

4. The method of claim 1, wherein the blood cancer is myeloma.

5. The method of claim 4, wherein the myeloma is selected from the group consisting of multiple myeloma, localized myeloma, plasmacytoma and extramedullary myeloma.

6. The method of claim 1, wherein the compound of formula (I) is in the form of a besylate salt.

7. The method of claim 1, wherein the therapeutically effective amount is from about 0.1 to about 10 milligrams.

8. The method of claim 7, wherein the therapeutically effective amount is from about 1 to about 4 milligrams.

9. The method of claim 8, wherein the therapeutically effective amount is from about 2 to about 3 milligrams.

10. The method of claim 9, wherein the therapeutically effective amount is about 3 milligrams.

11. The method of claim 1, wherein the pharmaceutical composition is administered to the subject twice a day, daily, every other day, three times a week, twice a week, weekly, every other week, twice a month, or monthly.

12. The method of claim 11, wherein the pharmaceutical composition is administered to the subject daily.

13. The method of claim 1, wherein the pharmaceutical composition is administered to the subject daily and the therapeutically effective amount of the compound is about 3 milligrams.

14. A method of treating the symptoms of leukemia in a subject in need thereof comprising administering to the subject a pharmaceutical composition comprising a therapeutically effective amount of a compound of formula (I), 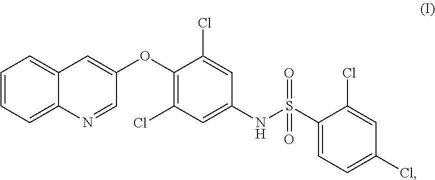 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

15. The method of claim 14, wherein the leukemia is selected from the group consisting of acute myelogenous leukemia, chronic myelogenous leukemia, acute lymphocytic leukemia, chronic lymphocytic leukemia, hairy cell leukemia, myelodysplastic syndromes and myeloproliferative disorders.

16. The method of claim 21, wherein the compound of formula (I) is in the form of a besylate salt.

17. The method of claim 21, wherein the therapeutically effective amount is from about 0.1 to about 10 milligrams.

18. The method of claim 17, wherein the therapeutically effective amount is from about 1 to about 4 milligrams.

19. The method of claim 18, wherein the therapeutically effective amount is from about 2 to about 3 milligrams.

20. The method of claim 19, wherein the therapeutically effective amount is about 3 milligrams.

21. The method of claim 21, wherein the pharmaceutical composition is administered to the subject twice a day, daily, every other day, three times a week, twice a week, weekly, every other week, twice a month, or monthly.

22. The method of claim 21, wherein the pharmaceutical composition is administered to the subject daily.

23. The method of claim 21, wherein the pharmaceutical composition is administered to the subject daily and the therapeutically effective amount of the compound is about 3 milligrams.

24. A method of treating the symptoms of myeloma in a subject in need thereof comprising administering to the subject a pharmaceutical composition comprising a therapeutically effective amount of a compound of formula (I), 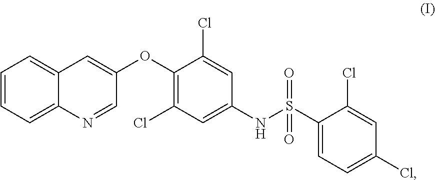 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

25. The method of claim 24, wherein the myeloma is selected from the group consisting of multiple myeloma, localized myeloma, plasmacytoma and extramedullary myeloma.

26. The method of claim 42, wherein the compound of formula (I) is in the form of a besylate salt.

27. The method of claim 42, wherein the therapeutically effective amount is from about 0.1 to about 10 milligrams.

28. The method of claim 27, wherein the therapeutically effective amount is from about 1 to about 4 milligrams.

29. The method of claim 28, wherein the therapeutically effective amount is from about 2 to about 3 milligrams.

30. The method of claim 29, wherein the therapeutically effective amount is about 3 milligrams.

31. The method of claim 42, wherein the pharmaceutical composition is administered to the subject twice a day, daily, every other day, three times a week, twice a week, weekly, every other week, twice a month, or monthly.

32. The method of claim 31, wherein the pharmaceutical composition is administered to the subject daily.

33. The method of claim 42, wherein the pharmaceutical composition is administered to the subject daily and the therapeutically effective amount of the compound is about 3 milligrams.

34. A method of treating a blood cancer selected from leukemia and myeloma in a subject in need thereof comprising increasing the serum adiponectin level in the subject by administering to the subject a pharmaceutical composition comprising a therapeutically effective amount of a compound of formula (I), 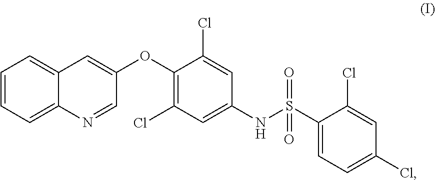 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

35. The method of claim 34, wherein the blood cancer is leukemia.

36. The method of claim 35, wherein the leukemia is selected from the group consisting of acute myelogenous leukemia, chronic myelogenous leukemia, acute lymphocytic leukemia, chronic lymphocytic leukemia, hairy cell leukemia, myelodysplastic syndromes and myeloproliferative disorders.

37. The method of claim 34, wherein the blood cancer is myeloma.

38. The method of claim 37, wherein the myeloma is selected from the group consisting of multiple myeloma, localized myeloma, plasmacytoma and extramedullary myeloma.

39. The method of claim 34, wherein the compound of formula (I) is in the form of a besylate salt.

40. The method of claim 34, wherein the therapeutically effective amount is from about 0.1 to about 10 milligrams.

41. The method of claim 40, wherein the therapeutically effective amount is from about 1 to about 4 milligrams.

42. The method of claim 41, wherein the therapeutically effective amount is from about 2 to about 3 milligrams.

43. The method of claim 42, wherein the therapeutically effective amount is about 3 milligrams.

44. The method of claim 34, wherein the pharmaceutical composition is administered to the subject twice a day, daily, every other day, three times a week, twice a week, weekly, every other week, twice a month, or monthly.

45. The method of claim 44, wherein the pharmaceutical composition is administered to the subject daily.

46. The method of claim 34, wherein the pharmaceutical composition is administered to the subject daily and the therapeutically effective amount of the compound is about 3 milligrams.

47. The method of claim 63, wherein the adiponectin level in the subject increases by at least about 30%, at least about 68%, at least about 175%, or at least about 200%.

48. The method of claim 47, wherein the adiponectin level in the subject increases by at least about 175%.

49. A method of treating a blood cancer selected from leukemia and myeloma in a subject in need thereof comprising increasing the serum adiponectin level in the subject by daily administering to the subject a pharmaceutical composition comprising about 1 to about 4 milligrams of a compound of formula (I), 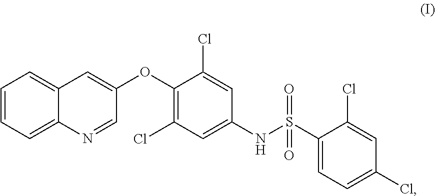 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

50. The method of claim 49, wherein the blood cancer is leukemia.

51. The method of claim 50, wherein the leukemia is selected from the group consisting of acute myelogenous leukemia, chronic myelogenous leukemia, acute lymphocytic leukemia, chronic lymphocytic leukemia, hairy cell leukemia, myelodysplastic syndromes and myeloproliferative disorders.

52. The method of claim 49, wherein the blood cancer is myeloma.

53. The method of claim 52, wherein the myeloma is selected from the group consisting of multiple myeloma, localized myeloma, plasmacytoma and extramedullary myeloma.

54. The method of claim 49, wherein the compound of formula (I) is in the form of a besylate salt.

55. The method of claim 49, wherein the therapeutically effective amount is from about 2 to about 3 milligrams.

56. The method of claim 55, wherein the therapeutically effective amount is about 3 milligrams.

57. The method of claim 49, wherein the pharmaceutical composition is administered to the subject daily and the therapeutically effective amount of the compound is about 3 milligrams.

58. The method of claim 63, wherein the adiponectin level in the subject increases by at least about 30%, at least about 68%, at least about 175%, or at least about 200%.

59. The method of claim 58, wherein the adiponectin level in the subject increases by at least about 175%.
